(19) United States
(12) Patent Application Publication
(10) Pub. No.: US 2018/0160976 A1
(43) Pub. Date: Jun. 14, 2018

(54) BIOSENSOR, TRANSPARENT CIRCUITRY AND CONTACT LENS INCLUDING SAME

(71) Applicant: UNIST (ULSAN NATIONAL INSTITUTE OF SCIENCE AND TECHNOLOGY), Ulsan (KR)

(72) Inventors: Jang Ung Park, Ulsan (KR); Min Ji Kim, Ulsan (KR); Joo Hee Kim, Ulsan (KR); Mi Sun Lee, Ulsan (US)

(73) Assignees: UNIST (ULSAN NATIONAL INSTITUTE OF SCIENCE AND TECHNOLOGY), Ulsan (KR); UNIST (ULSAN NATIONAL INSTITUTE OF SCIENCE AND TECHNOLOGY), Ulsan (KR)

(21) Appl. No.: 15/525,120

(22) PCT Filed: Sep. 15, 2015
(86) PCT No.: PCT/KR2015/009657

§ 371 Date: May 8, 2017

(30) Foreign Application Priority Data
Nov. 10, 2014 (KR) 10-2014-0155675

(51) Int. Cl. A61B 5/00; H01L 29/16; G02C 7/04; G02C 11/00; A61B 5/145; A61B 5/1486; G01N 27/327; H01Q 1/36

(52) U.S. Cl. CPC A61B 5/6821; H01L 29/1606; G02C 7/04; G02C 11/10; H01Q 1/273; A61B 5/1486; G01N 27/3271; H01Q 1/368; A61B 5/14532

(57) ABSTRACT

Lenses, including contact lenses, and other transparent substrates include electronic circuits having patterned conductors and antenna structures which are transparent, flexible and conductive. A patterned conductor or antenna structure can be a combination of two-dimensional material such as graphene and one-dimensional material such as metal nanowires. The patterned conductor or antenna structure can be wrinkled or otherwise pre-stressed, to accommodate stretching and folding of the substrate. A biosensor having a sensor unit and an antenna unit, or other type of circuit, can be formed using these materials, and can be disposed on a contact lens.

### TECHNICAL FIELD

The present technology relates to lenses having circuitry thereon, including biosensors, with transparent conductors and methods for manufacturing such devices.

### BACKGROUND ART

Indium tin oxide (ITO) is commonly used as a material for transparent electrodes and conductors for light-emitting diodes, touch screens and the like. However, ITO has a relatively high sheet resistance. Also, sources of supply in raw materials markets for indium are unstable. Also, the material is quite expensive.

Recently, researchers have been actively looking for transparent conductive materials that can be used as a substitute for ITO and for other applications requiring transparent conductors.

For example, technology has been developed that uses graphene as a transparent electrode. However, the sheet resistance of graphene is high. Therefore, transparent conductive materials that have a low sheet resistance while also retaining a high optical transmittance are in demand.

Also, it is desirable to provide transparent conductive materials and circuit structures suitable for use in electro-active contact lenses, other lens structures, epidermal electrodes and other devices where low visibility circuitry is desirable.

### DISCLOSURE OF INVENTION

### Technical Problem

The objective of the present invention is to provide lenses having circuitry thereon, including biosensors, with transparent conductors and methods for manufacturing such devices.

### Solution to Problem

A technology is described that provides patterned conductors and antenna structures which are transparent, flexible and conductive, suitable for use on lenses and other devices where low visibility circuitry is desirable, including, for example, contact lenses and epidermal electrode systems. According to one aspect, a patterned conductor or antenna structure is provided on a substrate that comprises a combination of two-dimensional nanomaterial such as graphene and conductive fibers that can be metal nanowires or other one-dimensional nanomaterial.

According to another aspect, the patterned conductor or antenna structure is electrically connected to circuit components on the substrate. In some embodiments, the patterned conductor or antenna structure is wrinkled or otherwise pre-stressed, to accommodate stretching and folding of the substrate on which it is disposed.

One objective of the technology presented here is to provide patterned conductors or antennas made of a transparent, flexible material suitable for use on or in a lens body substrate such as used for a contact lens, another type of lens or other substrates on which transparent circuitry is desirable. Other objectives include providing methods for manufacturing the same.

One additional objective of the technology presented here is to provide a biosensor having a sensor unit and an antenna unit, or other type of circuit, formed by using a nanomaterial and a method for manufacturing the same.

Other aspects and advantages of the technology described herein can be seen on review of the drawings, the detailed description and the claims, which follow.

### Advantageous Effects of Invention

A biosensor described herein has the advantage in which a high optical transmittance and flexibility and wireless communication with the outside are enabled by comprising an electrode and an antenna that are formed of graphene and a silver nanowire, and a channel formed of graphene only.

Further, there is an advantage in detecting a glucose concentration in tears while wearing contact lenses.

Further, there is an advantage that an electrode, patterned conductor and an antenna unit can be simultaneously manufactured together, and therefore, the manufacture is simple.

### BRIEF DESCRIPTION OF DRAWINGS

FIG. 1 is an illustration providing a perspective view of a biosensor including a patterned conductor and antenna structure on a contact lens as described herein.

FIGS. 2A, 2B and 2C illustrate stages in a manufacturing process for a biosensor like that shown in FIG. 1.

FIG. 3 is a cross-sectional view of materials used to form the patterned conductor and antenna structures for the biosensor shown in FIG. 1.

FIG. 4 is a cross-sectional view of the channel structure for the biosensor shown in FIG. 1.

FIG. 5 is a simplified flowchart of a manufacturing process which can be used to form the structure shown in FIG. 1.

FIG. 6 is a graph of current versus voltage showing performance of a biosensor with various concentrations of glucose.

FIG. 7 is a graph showing current versus time showing performance of a biosensor with various concentrations of glucose.

FIG. 8 is a graph showing changes in reflection coefficient of a circuit like that shown in FIG. 1 with glucose concentration.

FIG. 9 is an illustration showing the layout of a circuit on a lens substrate including patterned conductors and antenna structures as described herein.

FIG. 10 is a simplified illustration of a wrinkled patterned conductor layer which accommodates folding and stretching of a lens substrate for circuits like those represented by FIG. 9 and FIG. 1.

### MODE FOR THE INVENTION

A transparent, flexible patterned conductor and a transparent, flexible antenna are described comprising a combination of a two-dimensional material such as graphene and conductive fibers which can be one-dimensional material, such as nanowires, disposed in a network or mesh on the two dimensional material. The patterned conductor and/or the antenna can be disposed on a substrate having an optical region, such as a contact lens substrate. The patterned conductor and/or the antenna can be pre-stressed, or wrinkled, to accommodate stretching and folding of the substrate.

A biosensor disposed on a contact lens is illustrated herein which provides for a convenient noninvasive way to determine glucose concentration.

A biosensor device described herein comprises:

an electrode comprising a one-dimensional material and a two-dimensional material and a channel formed of a two-dimensional material; and

an antenna unit, comprising at least one of a one-dimensional material, a two-dimensional material, and a combination thereof.

Alternatively, a contact lens described herein comprises an electrode comprising a one-dimensional material and a two-dimensional material, and a channel formed of the two-dimensional material, and an antenna unit. The antenna unit comprises at least one of a one-dimensional material, a two dimensional material, and a combination thereof. The contact lens may comprise a biosensor as described herein.

The biosensor can be operated by disposing a reader within a predetermined distance of the biosensor, and exciting the antenna with an RF signal. The excited antenna can inductively couple current to a patterned conductor loop connected to the electrodes and to the active channel of the biosensor. A value of the current can be sensed by said reader by detecting a reflection value as an electromagnetic resonance with said sensor unit.

A biosensor described herein according to another aspect of the technology comprises:

a sensor unit comprising a patterned conductor connected to electrodes formed of a transparent and flexible two-dimensional material and a one-dimensional material and a channel formed of said two-dimensional material only between the electrodes;

an antenna unit spaced from said sensor unit for inductive coupling between the two, comprised of said two-dimensional material and said one-dimensional material, the combination capable of producing a signal based on induced electromagnetic resonance; and

a contact lens to which said sensor unit and said antenna unit are transferred.

The patterned conductor and antenna can respectively comprise a first graphene layer formed on a sacrificial substrate by a transfer method, conductive fibers such as metal nanowires coated on said graphene layer and overlapped with one another, thereby forming a network, and a second graphene layer formed on said first graphene layer and fibers by a transfer method. The channel can comprise a graphene layer positioned between both ends of said electrode and formed by a transfer method and an enzyme layer coated with a glucose oxidase on said graphene layer, said patterned conductor formed in a ring shape connecting to the first and second electrodes. The first and second electrodes have an opening formed to dispose said channel. The antenna is formed in a spiral shape inside or outside the loop formed by the patterned conductor.

A method for manufacturing a biosensor described herein comprises:

forming a first graphene layer by transferring graphene onto a sacrificial substrate;

forming a graphene-nanowire layer by coating conductive fibers such as nanowires on said first graphene layer;

forming electrodes, a patterned conductor and an antenna by patterning said graphene-nanowire layer in an electrode shape, patterned conductor shape and antenna shape, respectively;

forming a second graphene layer by transferring graphene onto said graphene-nanowire layer in which said electrodes, patterned conductor and antenna have been formed; and

patterning said second graphene layer to cross between the first and second electrodes to form a channel. Also, the second graphene layer can be patterned to match said electrode shape, said patterned conductor shape and said antenna shape, in addition to the channel shape.

A biosensor described herein has the advantage in which a high optical transmittance and flexibility and wireless communication with the outside are enabled by comprising an electrode and an antenna that are formed of graphene and a silver nanowire, and a channel formed of graphene only.

Further, there is an advantage in detecting a glucose concentration in tears while wearing contact lenses.

Further, there is an advantage that an electrode, patterned conductor and an antenna unit can be simultaneously manufactured together, and therefore, the manufacture is simple.

A detailed description of embodiments of the technology is provided with reference to the FIGS. 1-10.

FIG. 1 illustrates a lens 10, which can be a contact lens, which includes a circuit disposed on a lens body substrate 60, where the lens body substrate is a body of polymeric material that is in or is fashioned into the shape of the contact lens that can be placed on the eye. Other components, such as described herein can be disposed on or in that lens body substrate. The circuit is disposed on the substrate by being embedded in a passivation film 7 on the substrate 60. In alternatives, the circuit can be disposed on the substrate by attachment to an upper or lower surface of the substrate, by being partially or completely embedded within the material of substrate 60, or otherwise. Thus, the passivation film 7 may be omitted, depending on the materials and techniques utilized. The elements of the circuit in the embodiment shown in FIG. 1 include a biosensor 40, a patterned conductor 25 and an antenna 50. In this example, the biosensor 40 has a channel 30 disposed between a first electrode 20 and a second electrode 21. The first and second electrodes 20, 21 are nodes in the circuit including the patterned conductor 25 and the biosensor 40. The biosensor 40 can be a field effect device, and although not used in this embodiment, may include a third electrode (i.e. gate electrode) by which a bias voltage can be applied to the channel 30. A patterned conductor 25 is configured in a loop disposed in this example near the perimeter of the passivation layer 7, and connects the first electrode 20 to the second electrode 21. The antenna 50 is disposed in this example inside the loop formed by the patterned conductor 25, and configured in a spiral having three loops. In other embodiments, the antenna 50 can be disposed outside the loop formed by the patterned conductor 25, and have a different number of loops. It can be preferred that both the patterned conductor 25 and the antenna be disposed in a region on the lens substrate outside the optical zone of the lens. The optical zone of a contact lens is typically a region 5 to 10 millimeters in diameter positioned to lie in the field of view of the eye. The patterned conductor 25 and antenna 50 can have widths for example, in the range of 100 to 500 microns. The patterned conductor 25 and antenna 50 can have widths determined by the limits of the technology used to form the patterns, such as on the order of a micron or less. Also, widths greater than 500 microns could be used in some types of systems. Of course, the dimensions of the circuit elements can be adapted as needed for a particular use of the technology.

The patterned conductor 25, the antenna 50, or both, and the first and second electrodes 20, 21 comprise a combination of a two-dimensional nanomaterial and conductive fibers. The conductive fibers can be one-dimensional nanomaterial such as metal nanowire. The antenna 50 can comprise the same combination of materials as the patterned conductor 25, or a different combination as suits a particular implementation. Also, the first and second electrodes can comprise the same combination of materials as the patterned conductor 25, or a different combination as suits a particular implementation.

The combination of two-dimensional nanomaterial and conductive fibers utilized to form the patterned conductor 25, the antenna 50, or both, is substantially transparent to visible light making the circuit elements suitable for use on a lens. For example, at least a portion of the combination of two-dimensional material and conductive fiber used for one or both of the patterned conductor and the antenna (not including the contact lens substrate) can have a transmittance greater than 80% for green light, and similarly high transmittance across the visual range so as to be perceived by the user as substantially transparent. In some embodiments, the combination of two-dimensional material and conductive fiber used for the patterned conductor and the antenna (not including the contact lens substrate) can have a transmittance of about 93% or more for green light (near a wavelength of 550 nm), bases on. UV-vis-NIR spectroscopy (Cary 5000 UV-vis-NIR, Agilent).

Also, a combination of two-dimensional nanomaterial and conductive fiber utilized to form the patterned conductor 25, the antenna 50, or both, has relatively low sheet resistance, making it suitable for use as conductors and antennas for electronic circuits. In some embodiments, the sheet resistance of the patterned conductor, the antenna, or both, can be less than 50 ohms/sq. In one example embodiment, sheet resistance of the patterned conductor can be on the order of 30 ohms/sq.

The biosensor 40 in this example circuit has one or both of the electrodes 20, 21 formed using the same combination of two-dimensional nanomaterial and conductive fiber utilized to form the patterned conductor 25. The channel 30 of the biosensor 40 can comprise a single layer of two-dimensional nanomaterial, such as graphene, with an active enzyme embedded in the graphene which can react with glucose or other reactant materials in tear fluids to produce charge carriers. The production of charge carriers influences the resistance of the biosensor 40, and can be detected to indicate amounts of reactant materials.

Hard lens substrates or soft contact lens substrates made of any known lens material may be used. Preferably, the lens substrates are used as soft contact lenses or parts of contact lenses, and have water contents of about 0 to about 90 percent. More preferably, the lens substrates may be made of monomers containing hydroxy groups, carboxyl groups, or both, or be made from silicone-containing polymers, such as siloxanes, hydrogels, “conventional” hydrogels, silicone hydrogels, silicone elastomers and combinations thereof. Material useful for forming the lenses may be made by reacting blends of macromers, monomers, and combinations thereof along with additives such as polymerization initiators.

For the purposes of this description, a definition of two-dimensional materials can be taken from http://www.nature.com/subjects/two-dimensional-materials published by Nature Publishing Group, Macmillan Publishers Limited, 2015, reading, “Two-dimensional materials are substances with a thickness of a few nanometers or less. Electrons in these materials are free to move in the two-dimensional plane, but their restricted motion in the third direction is governed by quantum mechanics. Prominent examples include quantum wells and graphene.” Such materials typically have a molecular structure which extends in only two dimensions.

For the purposes of this description, a definition of one-dimensional material can be a fiber having a thickness or diameter constrained to tens of nanometers or less. One-dimensional materials as the term is used herein can also be called nanowires. See, for example, “Nanowire,” Wikipedia, The Free Encyclopedia; date retrieved: 10 Feb. 2015 22:01 UTC, (http://en.wikipedia.org/w/index.php?title=Nanowire&oldid=641223222). Conductive fibers, such as metal nanowires, can be one-dimensional materials, if they have thicknesses on the order of tens of nanometers or less.

One combination of two-dimensional materials and conductive fibers having a good transmittance in the visible range, including transmittance greater than 90% for green light near 550 nm wavelength, and a sheet resistance less than 50 ohms per square, which is also flexible and thereby suitable for use on soft contact lens substrates, includes a layer of graphene and a mesh of conductive fibers like silver nanowires. The layer of graphene provides a strong two-dimensional lattice structure which is relatively conductive, transmissive and flexible. The mesh of conductive fibers, such as for example metal nanowires having diameters of about 30+/−5 nm with lengths of about 25+/−5 μm, forms a composite mesh of interconnections across the graphene layer for conduction of electricity. In some embodiments, the conductive fibers can have diameters in the range of 20 nm to 100 nm, and lengths up about 100 μm. Although longer and thicker conductive fibers are preferred to reduce the sheet resistance of the films, suspension of the conductive fibers in liquid solvents can be limited for larger fibers. In one example, silver nanowires are utilized. Other metals, such as platinum, gold and copper, and combinations of metals can be utilized. The density of the conductive fibers is such that the combination remains transmissive, conductive and flexible. In some embodiments, a second layer of graphene is disposed over the mesh of conductive fibers for added strength and conductivity.

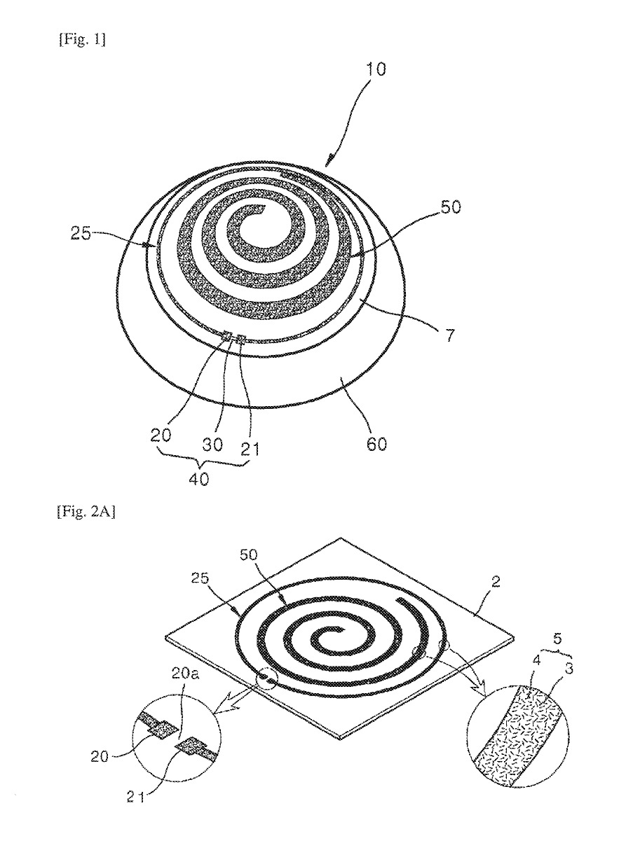

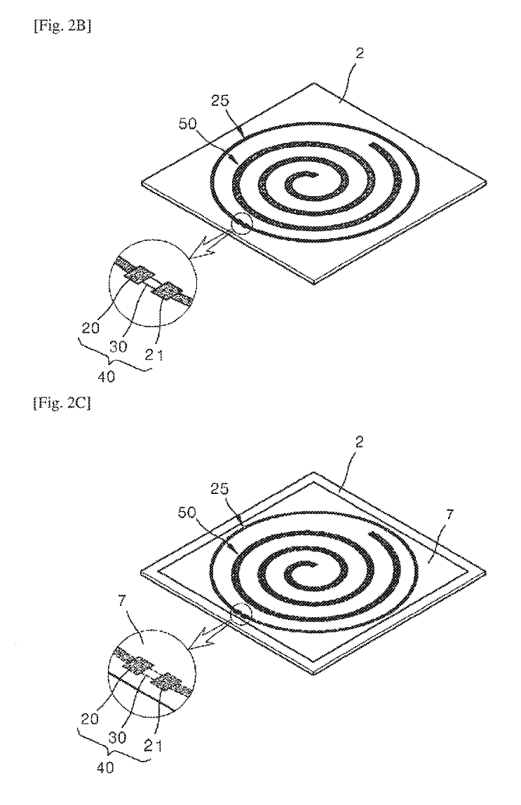

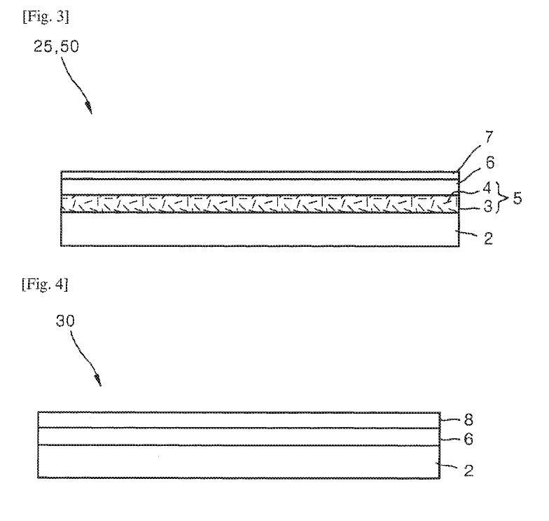

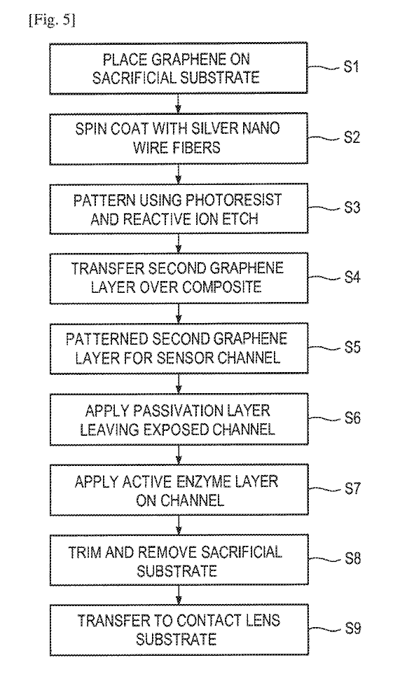

FIGS. 2A, 2B and 2C illustrate stages in a process for manufacturing a circuit which can be disposed on a lens substrate like that illustrated in FIG. 1. FIGS. 3 and 4 are cross-sectional views of portions of the structure shown in FIGS. 2A, 2B and 2C. FIG. 5 is a simplified flowchart of a manufacturing method.

FIG. 2A illustrates a sacrificial substrate 2 on which the patterned conductor 25, antenna 50 and electrodes 20 and 21, with a gap 20a therebetween, are formed, using a combination of a graphene layer and a mesh of conductive fibers. The graphene layer can be formed by growth on a copper foil with methane and hydrogen gas utilizing known techniques. The graphene layer on the copper foil is transferred onto a supporting layer by for example spin coating poly(methyl methacrylate PMMA) (MicroChem Corp. 950 PMMA) on the graphene. Then the copper foil is floated on a diluted etchant (e.g. FeCl₃:HCl:H₂ at 1:1:20 vol % ratios) and then etched. A PMMA coated graphene layer results. The graphene layer can then be cleaned with deionized water and transferred onto a chosen sacrificial substrate 2. The PMMA material can be removed by acetone.

A mesh of conductive fibers can be formed by suspending the fibers in a solution, and spin coating the solution over the graphene layer. In one example, 3 mg/mL silver nanowires (30+/−5 nm diameter and 25+/−5 μm long) were dispersed in deionized water stored at 5° C., and stirred at room temperature before spin coating. The solution was spun at 500 rpm for 30 seconds. After spin coating the material onto the graphene layer, the structure can be annealed to evaporate the solvent, for example.

The pattern illustrated in FIG. 2A can be defined using a photolithographic process, including applying a photoresist, and patterning the photoresist and etching the combination of conductive fibers and graphene using reactive ion etching or other etching processes. As mentioned above, the pattern in this example includes a patterned conductor 25 which extends from a first circuit node at the electrode 20 in a loop disposed near the perimeter of the lens to a second circuit node at the electrode 21. An antenna 50 is disposed inside the loop formed by the patterned conductor 25 and configured in a spiral shape. The electrodes 20, 21, the patterned conductor 25, and the antenna 50, comprise a graphene layer 3 and a mesh 4 of conductive fibers, which together form a conductive, transparent and flexible composite material layer 5. FIG. 2B illustrates a stage in the process after formation of a second graphene layer over the structure and patterning the second graphene layer. Thus, a second graphene layer is formed, such as using the copper foil process discussed above, and transferred onto the sacrificial substrate 2 over the circuit comprising the electrodes 20, 21, the patterned conductor 25, and the antenna 50. After transferring onto the substrate 2, the second graphene layer is patterned using a lithographic process to form a channel 30 in the biosensor which consists of a single graphene layer. Also, the second graphene layer can overlie the circuit including the electrodes 20, 21, the patterned conductor 25, and the antenna 50, adding a second graphene layer to the composite.

FIG. 2C illustrates a stage in the process after formation of a passivation film layer 7 over the circuitry on the sacrificial substrate 2. The passivation layer can be formed using any suitable transparent and flexible polymer. For example, in one embodiment, the passivation layer comprises a photoresist (e.g. SU8) which can be patterned to expose the channel region 30 on the biosensor 40, and developed to form a passivation layer 7 over the balance of the structure. Other materials can be used as the passivation layer, such as parylene, PDMS, SiO2, and so on. It is desirable that the passivation material be transparent, insulating, biocompatible.

In the exposed region over the channel 30, an enzyme layer 8 is formed using for example glucose oxidase in combination with a pyrene linker used as a connecting material bonding graphene to the glucose oxidase.

Then, the passivation layer and sacrificial substrate 2 are cut in a circular pattern in a shape suitable to be transferred onto the contact lens substrate. The sacrificial substrate 2 can be removed prior to transfer. FIG. 1 illustrates a resulting structure including a circuit on a lens substrate within a passivation film layer 7.

FIG. 3 is a heuristic cross-sectional view of the stack of materials used to form the patterned conductor 25 and the antenna 50 as shown in FIG. 2C. The stack includes the sacrificial substrate 2, a composite layer 5 (including the first graphene layer 3, and the mesh of conductive fibers 4), and a second graphene layer 6. Passivation layer 7 overlies the second graphene layer. The view is not drawn to scale. The transmittance (transparency) and the sheet resistance (conductivity) of the structure can be adjusted by varying the length, density and type of conductive fibers and the number of graphene layers utilized. It is desirable for the material used for patterned conductor and antenna applications to have sheet resistance less than 50 ohms/square. It is desirable for lens applications, or other applications on transparent substrates, for the transmittance of the combination of two-dimensional material and conductive fiber to be greater than 80% over at least most of the visible range, as discussed above. A stack of materials as shown in FIG. 3 has a high elasticity and is highly flexible, and can be bent around a radius of a few microns with resulting induced strain, without significant change in resistance.

FIG. 4 is a heuristic cross-sectional view of the stack of materials used to form the channel 30 in the biosensor. The stack and the channel region include the sacrificial substrate 2, the second graphene layer 6, and an enzyme layer 8 such as a glucose oxidase and a linker material. The view is not drawn to scale.

FIG. 5 is a simplified flowchart summarizing an example of the manufacturing process just described with reference to FIGS. 1 through 4. The example process includes placing a graphene layer on a sacrificial substrate (S1). Then, silver nanowire fibers are spin coated over the graphene layer (S2). The composite is patterned using photoresist and reactive ion etching (S3). Next, a second graphene layer is transferred over the patterned composite (S4). A second patterning step is applied to match the second graphene layer with the pattern of the patterned composite, and to form a sensor channel (S5). The passivation layer is applied leaving the channel exposed (S6). An active enzyme layer is applied on the channel (S7). The passivation layer is trimmed and the sacrificial substrate is removed (S8). The circuit is then transferred to a contact lens substrate (S9).

As with all flowcharts herein, it will be appreciated that many of the steps can be combined, performed in parallel or performed in a different sequence without affecting the functions achieved. In some cases, as the reader will appreciate, a rearrangement of steps will achieve the same results only if certain other changes are made as well. In other cases, as the reader will appreciate, a rearrangement of steps will achieve the same results only if certain conditions are satisfied. Furthermore, it will be appreciated that the flow charts herein show only steps that are pertinent to an understanding of the invention, and it will be understood that numerous additional steps for accomplishing other functions can be performed before, after, and between those shown.

In this example, the biosensor is configured to sense glucose in tear fluid while a contact lens is being worn. Glucose in the tear fluid from the patient's eye reacts with the glucose oxidase on the channel 30. After oxidation of the glucose by the glucose oxidase, reduced glucose oxidase can be oxidized by reaction with oxygen forming hydrogen peroxide as a by-product. This hydrogen peroxide is also oxidized into water generating charge carriers. The circuit can be excited using an external radiofrequency RF source tuned near the resonant frequency of the structure. In one example, the resonant frequency can be in the range of 3 GHz to 4 GHz. The conductivity of the biosensor is a function of the glucose concentration, and impacts the reflection coefficient (e.g. the S11 parameter) of the circuit. This reflection coefficient can be measured to indicate glucose concentration in the tear fluid.

Other reactant materials can be placed in the channel region to sense different types of materials or different conditions in the tear fluid or on the lens.

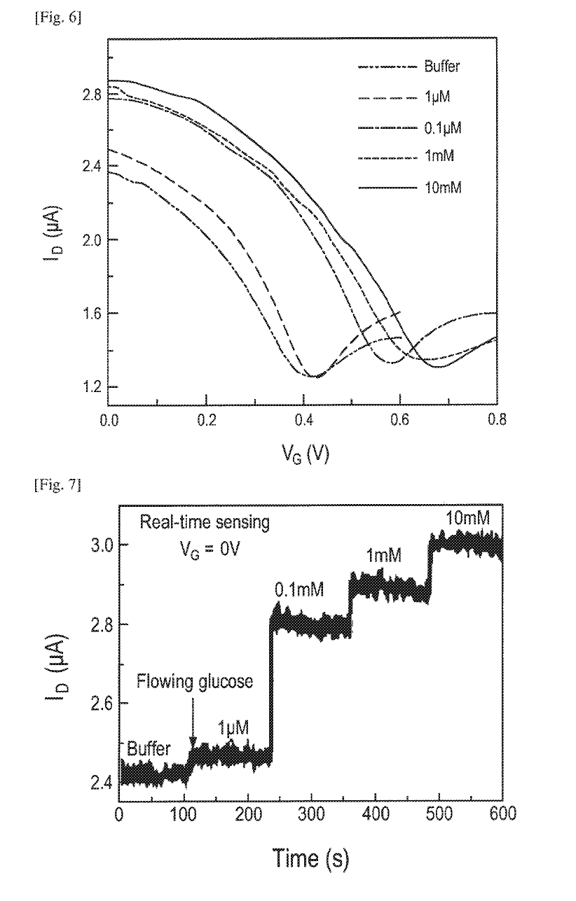

FIG. 6 is a graph of drain current versus gate voltage for a field effect device including a graphene channel and composite electrodes such as those described with reference to FIG. 1, configured for glucose sensing. The graph illustrates sensing of five samples ranging from a buffer control sample, from 1 microMole per liter (μM) of glucose to 10 milliMoles per liter (mM) of glucose, demonstrating that the drain current in such a field effect device is a function of the glucose concentration. A higher glucose concentration results in a higher drain current in the tested device.

FIG. 7 is a graph of drain current versus time with a gate voltage at 0 V, as a fluid is flowed across the channel region with changing concentrations. This graph illustrates the same information as shown in FIG. 6. Also, the graph shows that the change is very fast, generated in real time.

The circuit shown in FIG. 1 including the biosensor 40 and the patterned conductor 25 can be characterized as a first resistance, inductance, capacitance RLC circuit on the lens substrate, having a resonant frequency. The antenna 50 constitutes a second RLC circuit which is inductively coupled with the first RLC circuit on the lens substrate. A reader including a third RLC circuit can be placed in proximity to the lens substrate to sense a reflection, and determine the reflection coefficient, in the presence of a radiofrequency stimulus. The reader can determine the glucose concentration from the sensed reflection coefficient.

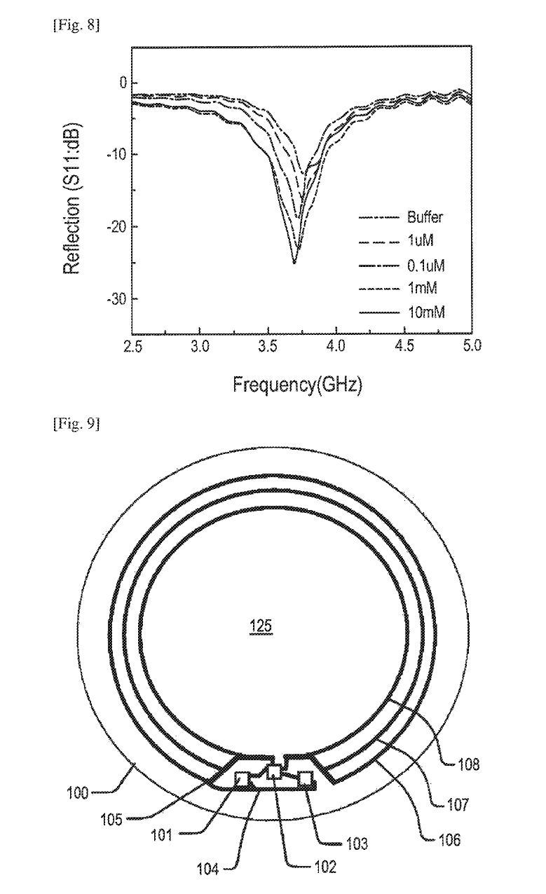

FIG. 8 is a graph showing a reflection coefficient measured according to glucose concentration for a circuit having an antenna made of an electrically conducting optically transparent graphene-silver nanowire hybrid that includes a spiral with three turns and a width of about 500μ, disposed outside a conductive loop connected to the biosensor, the conductive loop having a width of about 120μ. The tests were conducted at glucose concentrations ranging from 1 microMole per liter to 10 milliMoles per liter. As illustrated in FIG. 8, the change in reflection coefficient S11 over the range of samples illustrated can be easily determined using an electronic reader circuit.

FIG. 9 illustrates another embodiment of a lens that includes a circuit on a lens substrate 100. In this example, the circuit includes electrical components 101, 102, 103 which can, for example, comprise a controller logic chip, a capacitor, a switch, a biosensor, or other circuitry elements. The circuit surrounds an optical region 125 on the lens substrate 100. A tunable optic or an electroactive lens, or elements thereof, can be disposed in the optical region 125 in some embodiments. A biosensor can be disposed in the optical region 125 and elsewhere on the substrate, in some embodiments. A biosensor can include a sensor that drives or controls a tunable optic or chemical sensor in some embodiments. The electrical components 101, 102, 103 have nodes that are electrically connected to patterned conductors (e.g. 104, 105) which interconnect the circuit elements to form the circuit. Also, one or more antenna loops 106, 107, 108 are electrically connected to the electrical components by conductive, capacitive or inductive electrical connections. One or more of the patterned conductors (e.g. 104, 105) and the antenna loops 106, 107, 108 comprises a combination of two-dimensional materials and conductive as described above. For example, the patterned conductors and antenna loops can comprise a composite of graphene and conductive fibers which is transparent, conductive and flexible.

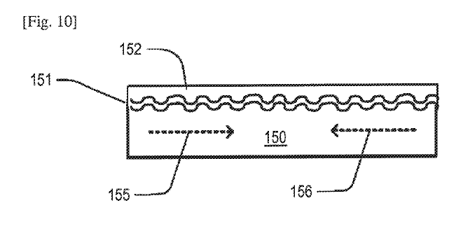

FIG. 10 illustrates aspects of an embodiment of the patterned conductors and antenna materials which have wrinkles, or are otherwise pre-stressed, to accommodate stretching of the lens substrate 100. The structure in FIG. 10 is a heuristic diagram of a sacrificial substrate 150, a wrinkled layer 151 that comprises a composite of a two-dimensional material and conductive fibers which is patterned to form a conductor or antenna, and a passivation layer 152 overlying the wrinkled layer 151. The structure can be formed by pre-stretching the sacrificial substrate 150 (which may comprise for example polydimethylsiloxane PDMS) in at least one direction and preferably two directions, and then forming the patterned conductor or antenna material on the pre-stretched sacrificial substrate 150. In one example, the substrate 150 can be pre-stretched by about 10%. Then the pre-stretched sacrificial substrate 150 is allowed to relax as represented schematically by the arrows 155, 156. The relaxation of the sacrificial substrate 150 causes wrinkling of the patterned conductor or antenna material to form the wrinkled layer 151. A flexible and stretchable passivation layer 152 (which may comprise for example polyethylene terephthalate PET) overlies the wrinkled layer 151. The passivation layer 152 can be applied before or after the sacrificial layer is allowed to relax. The sacrificial substrate 150 can be removed and the wrinkled structure transferred to the foldable lens substrate 100 using techniques as described above. In one example, a conductors formed in this manner have been tested under up to 10% tensile strain, with negligible changes in resistance.

A lens substrate is described having a patterned conductor, an antenna, or both, thereon having wrinkles to accommodate stretching, folding, or both, of the lens substrate. This technique can be applied as well to the patterned conductor and antenna of the biosensor circuit described with reference to FIG. 1. In addition, this technique can be applied to the patterned conductors and antenna structures shown in FIG. 9 for connection to other types of circuits.

Using the wrinkled combination of two-dimensional material and one-dimensional material, the circuit is very flexible and bendable, accommodating folding of the lens substrate 100 while maintaining quality electrical performance, avoiding stress on connections to electrodes in the circuit, and maintaining transparency.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims.

1. A device comprising:
a substrate;
a circuit having first and second circuit nodes disposed on the substrate; and
a patterned conductor disposed on the substrate connecting the first and second circuit nodes, wherein the conductor comprises a combination of two-dimensional material and conductive fiber.

2. The device of claim 1, wherein at least a portion of the combination of two-dimensional material and conductive fiber of the conductor has a transmittance greater than 80% for green light.

3. The device of claim 1, wherein the circuit includes a field effect device having first and second electrodes, wherein said first electrode is said first circuit node.

4. The device of claim 1, wherein the two-dimensional material is graphene.

5. The device of claim 1, wherein the conductive fiber is disposed in the form of a mesh on the two-dimensional material.

6. The device of claim 1, including an antenna, wherein the antenna comprises said combination of two-dimensional material and conductive fiber.

7. The device of claim 1, wherein said combination of two-dimensional material and conductive fiber has wrinkles which accommodate stretching or folding of the substrate.

8. The device of claim 7, wherein said substrate is a foldable lens substrate.

9. The device of claim 1, wherein said combination of two-dimensional material and conductive fiber includes a first graphene layer, a mesh of conductive fibers disposed on the graphene layer, and a second graphene layer disposed on the mesh.

10. The device of claim 1, wherein said first and second nodes comprise electrodes, the electrodes comprising said combination of two-dimensional material and conductive fiber.

11. The device of claim 1, wherein said circuit comprises a biosensor.

12. The device of claim 1, wherein said circuit comprises a tunable optic.

13. The device of claim 1, wherein said circuit comprises an electroactive lens.

14. The device of claim 1, wherein said substrate is a lens substrate having an optical region.

15. A device comprising:
a substrate;
a circuit on the substrate; and
an antenna on the substrate electrically coupled with the circuit, the antenna comprising a combination of two-dimensional material and conductive fiber.

16. The device of claim 15, wherein at least a portion of the combination of two-dimensional material and conductive fiber of the antenna has a transmittance greater than 80% for green light.

17. The device of claim 15, wherein the circuit includes a field effect device having first and second electrodes.

18. The device of claim 15, wherein the two-dimensional material is graphene.

19. The device of claim 15, wherein the conductive fiber is disposed in the form of a mesh on the two-dimensional material.

20. The device of claim 15, wherein said combination of two-dimensional material and conductive fiber has wrinkles which accommodate stretching of the substrate.

21-87. (canceled)
